(19) United States
(12) Patent Application Publication
(10) Pub. No.: US 2007/0161942 A1
(43) Pub. Date: Jul. 12, 2007

(54) BIODEGRADABLE TAMPON APPLICATOR AND METHOD OF MAKING

(75) Inventors: Vancho Cirovski, Tecumseh (CA); David Kirby, Tecumseh (CA); John Mikhail, Tecumseh (CA)

Correspondence Address: Thomas Moga;Butzel Long, STONERIDGE WEST, 41000 WOODWARD AVENUE, BLOOMFIELD HILLS, MI, 48304, US

(73) Assignee: PHARMAPHIL INC., Tecumseh (CA)

(21) Appl. No.: 11/619,853

(22) Filed: Jan. 4, 2007

Related U.S. Application Data
(60) Provisional application No. 60/757,050, filed on Jan. 6, 2006.

(51) Int. Cl. A61F 13/20

(52) U.S. Cl. 604/15

(57) ABSTRACT

A non-cellulose, non-polymerized biodegradable tampon applicator and method of manufacture are provided. The tampon applicator includes a cap and a base that are formed on pins by dipping. The cap and base are dissolvable within minutes of exposure to water.

### TECHNICAL FIELD

The present invention relates generally to applicators for tampons and to a method of fabricating tampon applicators. More particularly, the present invention relates to a non-cellulose, non-polymerized biodegradable tampon applicator having cap and end components that are formed on pins.

### BACKGROUND OF THE INVENTION

Applicators have been long used for placement of a tampon. Once the tampon is in place the applicator is thrown away as it is not practically reusable. Given this circumstance for some time many applicators have been composed of a cellulose-based material to make them biodegradable. However, the cellulose-based material, while being environmentally attractive, has proven hard to form and uncomfortable to use

As an alternative to the cellulose-based material, polymerizable materials have been selected by many manufacturers. Polymerizable materials are relatively easy to form, can be used at a low cost, and can be readily shaped to form a smooth, user-friendly configuration. While overcoming the known disadvantages of cellulose-based material, the polymerized tampon applicators present significant environmental challenges. While the polymerized tampon applicator is thrown away as is the cellulose-based applicator, the latter biodegrades relatively quickly while the former is not readily given to biodegradation.

An alternative to both the cellulose-based applicator and to the polymerized applicator is desired. The present invention presents an applicator composed of a biodegradable material that has the forming and configuration benefits of polymerized material applicators but with environmental compatibility.

### SUMMARY OF THE INVENTION

The present invention overcomes the problems related to the prior art by providing a biodegradable tampon applicator that is easy to manufacture, uses non-cellulose, non-polymerized materials, and is easy to use.

The cap and base are formed from a composition of gelatin, silicon dioxide, sodium lauryl sulphate, and water. The composition is heated until it achieves a desired viscosity. Once the correct level of viscosity is established, pin bars are dipped into the flowable composition. The pin bars and the coated composition are allowed to cool. Once cooled, the pin bars are stripped and the cap and base elements of the applicator are attached with a pledget inserted therebetween.

Two embodiments of the present invention are taught. In the first embodiment, the base of the tampon applicator is separated from the cap. The pledget remains inside the cap. The base is reversed, the pledget withdrawing string is inserted through an aperture defined in the end of the base, and the base is re-inserted, end-first, into the cap. The user then can push the pledget through the petal tips formed at the insertion end of the cap by applying force to the base.

In the second embodiment of the present invention, no separation of the base from the cap is needed. Instead, the user applies insertion pressure on the base, forcing the pledget through the petal tips.

Regardless of the embodiment, the cap and the base may be disposed in the toilet. Each component is biodegradable and will readily degrade within minutes after contact with water.

The present invention provides several advantages over known applicators. First, the water-soluble, biodegradable materials used in the present invention are environmentally friendly and do not pose either chemical or physical harm to sewage systems. Second, the cap and base of the tampon applicator of the present invention are readily easy to make. Third, the costs associated with formulating, producing, assembly and packaging the tampon applicator of the present invention are relatively low.

Other advantages and features of the invention will become apparent when viewed in light of the detailed description of the preferred embodiment when taken in conjunction with the attached drawings and the appended claims.

### BRIEF DESCRIPTION OF THE DRAWINGS

For a more complete understanding of this invention, reference should now be made to the embodiment illustrated in greater detail in the accompanying drawings and described below by way of examples of the invention wherein:

FIG. 1 shows a perspective view of a pin for making the cap of the tampon applicator according to the present invention;

FIG. 2 shows a perspective view of a pin for making a preferred embodiment of the base of the tampon applicator of the present invention;

FIG. 3 shows a perspective view of a pin for making an alternate embodiment of the base of the tampon applicator of the present invention;

FIG. 4 illustrates a side view of a tampon applicator according to the preferred embodiment of the present invention;

FIG. 5 illustrates a sectional view of the tampon applicator illustrated in FIG. 4 showing the applicator elements in their initial, pre-application arrangement;

FIG. 6 illustrates a sectional view of the tampon applicator illustrated in FIGS. 4 and 5 but showing the base having been reversed for insertion into the applicator cap;

FIG. 7 illustrates a side view of a tampon applicator according to the alternate embodiment of the present invention; and

FIG. 8 illustrates a sectional view of the tampon applicator illustrated in FIG. 7.

### DETAILED DESCRIPTION OF THE PREFERRED EMBODIMENT

In the following figures, the same reference numerals will be used to refer to the same components. In the following description, various operating parameters and components are described for one constructed embodiment. These specific parameters and components are included as examples and are not meant to be limiting.

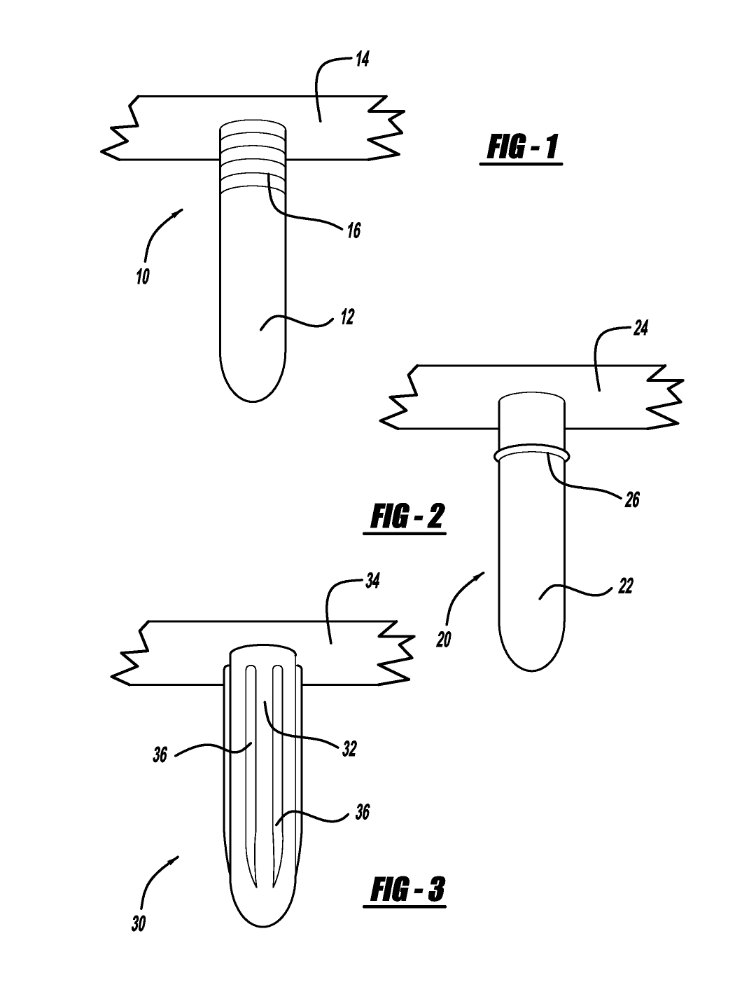

Referring now to FIG. 1, a perspective view of a pin bar assembly, generally illustrated as 10, is shown. The pin bar assembly 10 includes a pin 12 and a bar 14. The pin 12 includes a series of peripheral rings 16 formed near its base. The pin 12 and the bar 14 are structured in a manner known in the art of pin bar manufacture. The pin 12 shown in FIG. 1 is suitable for forming a cap for use in the tampon applicator of the present invention as will be discussed below.

Referring to FIG. 2, a perspective view of another pin bar assembly, generally illustrated as 20, is shown. The pin bar assembly 20 includes a pin 22 and a bar 24. The pin 22 includes a peripheral ring 26 formed near its base. The pin 22 shown in FIG. 2 is suitable for forming a base according to a first embodiment of the present invention as will be discussed below.

Referring to FIG. 3, a perspective view of another pin bar assembly, generally illustrated as 30, is shown. The pin bar assembly 30 includes a pin 32 and a bar 34. The pin 32 has a series of grooves 36 formed externally along the long axis of the pin 32. The pin 32 shown in FIG. 3 is suitable for forming a base according to a second embodiment of the present invention as will be discussed below.

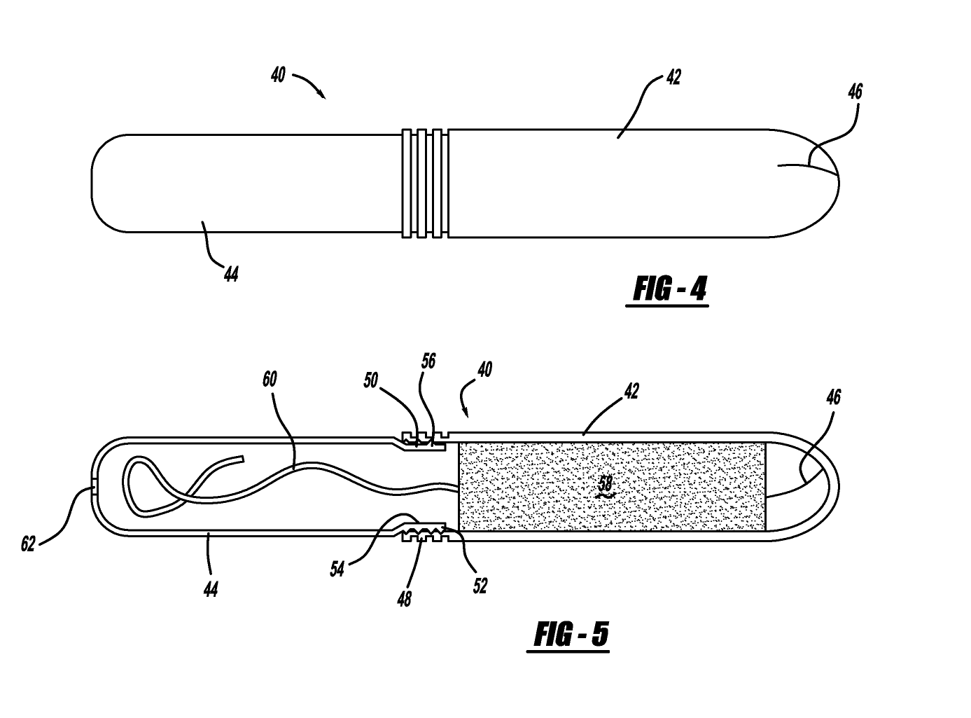

With reference now to FIG. 4, a perspective view of a biodegradable tampon applicator according to the first embodiment of the present invention, generally referred to as 40, is disclosed. The applicator 40 includes a cap 42 formed on the pin 12 shown in FIG. 1 and a base 44 formed on the pin 22 shown in FIG. 2. The end of the cap 42 has been cut at notch 46 (and others, not seen) to allow passage of the pledget thereby upon insertion. The cap 42 and the base 44 are shown in their inter-attached, pre-use positions.

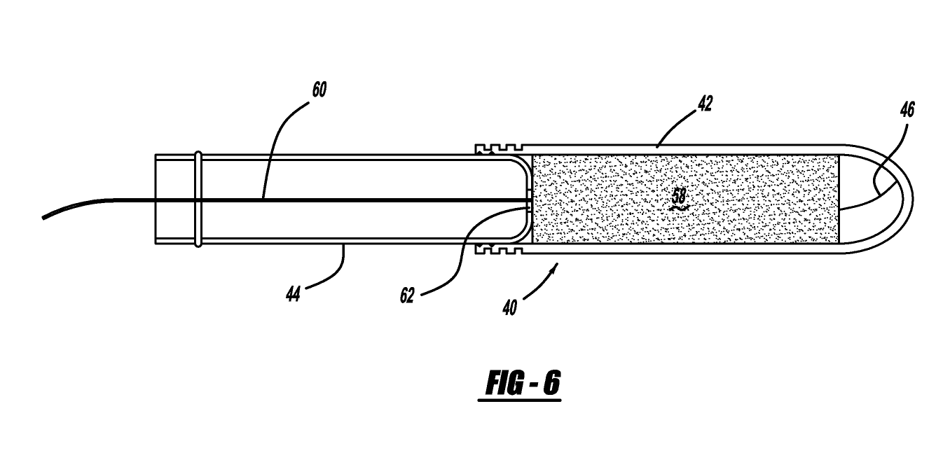

FIG. 5 illustrates a cross-sectional view of the tampon applicator 40 shown in FIG. 4. As may be understood by reference to the illustration, the cap 42 includes an open end 48 which has formed thereabout a series of parallel peripheral rings 50. The rings 50 are formed by the rings 16 of the pin 12 shown in FIG. 1.

The rings 50 serve a dual purpose with respect to the embodiment of the tampon applicator illustrated in FIGS. 4 through 6. The first use is to provide finger grips for the user upon manipulation of the base 44 with respect to the cap 42 when the pledget is inserted in a known manner. The second use is to provide a method for releasably locking the base 44 within the cap 42.

To achieve the second purpose, the rings 50 define a like number of inner channels 52. Similarly, the base 44 has an open end 54 which has formed thereabout a peripheral ridge 56. The ring 56 is formed by the ring 26 of the pin 22. As illustrated, the ring 56 of the base 44 generally nests within and thereby engages one of the inner channels 52 in a releasably lockable manner.

Substantially provided within the cap 42 of the applicator 40 is a pledget or absorbent device 58. The pledget 58 is formed from absorbent material that is known to those skilled in the art. Extending from one end of the pledget 58 is a withdrawing string 60, the arrangement and construction of which is also known to those skilled in the art.

FIG. 6 illustrates a view of the applicator 40 similar to that shown in FIG. 5 but showing the base 44 having been reversed and re-positioned within the open end 48 of the cap 42. Prior to insertion, the user removes the base 44 from the cap 42, reverses the cap 42, and places the free end of the withdrawing string 60 into and through an aperture 62 defined in the closed end of the cap 42. The user then re-inserts the base 44, closed-end first, into the open end 48 of the cap 42 until the closed end of the base 44 engages an end of the pledget 58. After the user inserts the rearranged applicator 40, the pledget 58 is pushed into position in a known manner. The user can then dispose of both the cap 42 and the base 44 in an available toilet. The cap 42 and the base 44, once exposed to water, degrade generally within about 10 minutes.

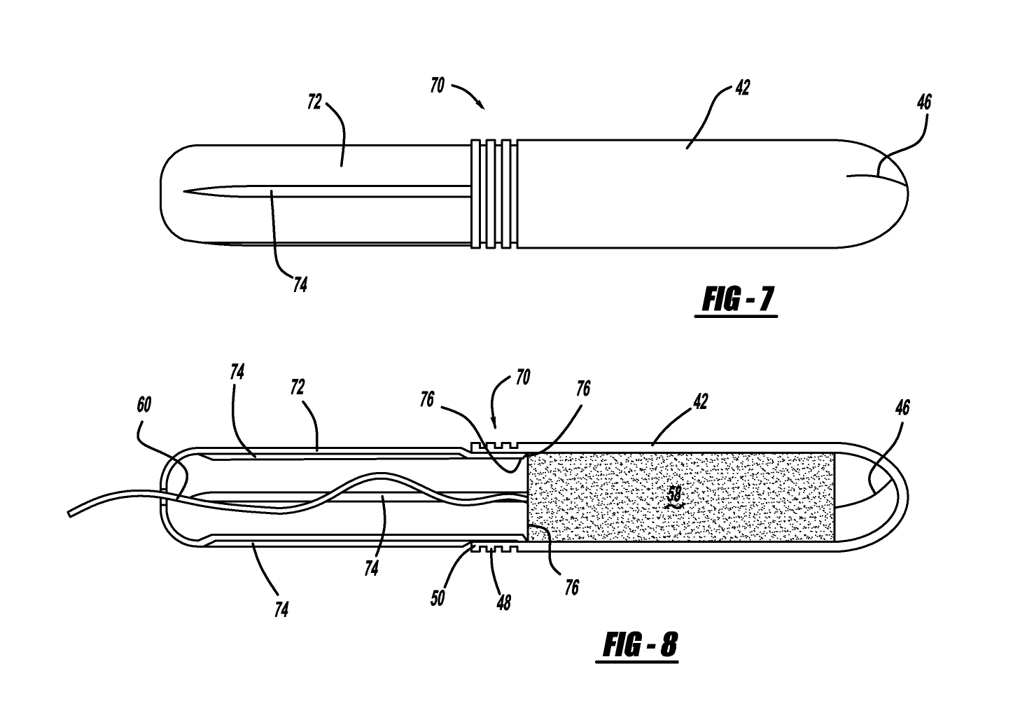

FIGS. 7 and 8 relate to an alternate embodiment of the biodegradable tampon applicator according to the present invention. As illustrated in FIG. 7, a tampon applicator, generally illustrated as 70, is shown. The tampon applicator 70 includes the cap 42 (as set forth above with respect to the embodiment of FIGS. 4-6) formed on the pin 12 shown in FIG. 1 and a base 72 formed on the pin 32 shown in FIG. 3. As set forth above, the cap 42 has been cut at notch 46 (and others, not seen) to allow passage of the pledget thereby.

FIG. 8 illustrates a cross-sectional view of the tampon applicator 70 shown in FIG. 7. As set forth above with respect to FIG. 5, the cap 42 includes the open end 48 which has formed thereabout the series of parallel peripheral rings 50. Also as set forth above, the rings 50 are formed by the rings 16 of the pin 12 shown in FIG. 1. The rings 50 are used here for gripping by the user upon manipulation of the base 72 with respect to the cap 42 when the pledget is inserted in a known manner.

The base 72 includes an array of interiorly-extending ridges 74 defined along its long axis. The ridges 74 are formed by the grooves 36 of the pin 32 shown in FIG. 3. The base 72 also includes an open end 76. Each of the ridges 74 terminates in a shelf or ledge 76 formed near the open end 76 of the base 72.

Substantially provided within the cap 42 of the applicator 70 is the pledget 58 described above. Attached to the pledget 58 is the withdrawing string 60, also described above. Unlike the tampon applicator 40 shown and described with respect to FIGS. 4 through 6 above, the tampon applicator 70 illustrated in FIGS. 7 and 8 does not need to be disassembled and then reassembled to operate. Instead, the shelves 76 are fitted against the string end of the pledget 58 when provided to the user. In operation, once substantially inserted, the user grips the rings 50 and applies pressure to the base 72 to insert the pledget 58 in a known manner.

Once the insertion operation is completed, the user can then dispose of both the cap 42 and the base 72 in an available toilet as set forth above with respect to the tampon applicator 40. The cap 42 and the base 72 are dissolved within about 10 minutes once exposed to water.

### Composition of the Tampon Applicator Cap and Base

Unlike known tampon applicators, the applicator of the present invention that has been described above is not made of paper or any cellulose-based material. Nor is it made of a polymerized material. Instead, the present invention is composed of a biodegradable composition that dissolves readily in water leaving no appreciable trace materials to cause a negative environmental impact. In addition, because the material of the present invention dissolves so readily upon contact with water, dissolution is relatively complete and no blockage to sewer systems should be anticipated.

The preferred composition of the present invention is as follows. Note that the quantities are preferred but are not intended as being limiting.

### Composition Components

| Component | Percentage |
|---|---|
| Gelatin | Between about 30.00% and 40.00% |
| Silicon dioxide | Between about 2.00% and 5.00% |
| Sodium lauryl sulphate | Between about 0.010% and 0.050% |
| Water | Between about 67.99% and 54.95% |

In addition, other components may be added to achieve a desired color or product texture. Specifically, in the event that a dye is added for coloration, 1.3% to 3.3% titanium dioxide (per 100 kg tank) may be added to improve adherence of the dye. (No dye or titanium dioxide would ordinarily be added in the event that a natural (clear) product is desired.) As a further addition, a preservative such as methylparaben may be included if desired in a known manner and in a known quantity.

Components may also be exchanged with other components as may be needed depending on local rules and regulations. For example, sodium lauryl sulphate may be substituted for by using a like amount of lecithin (80.0 grams per 100 kg tank), thus resulting in a sodium lauryl sulphate-free composition. By way of further example, gelatin may also be substituted for by using a gelatin alternative, such as cellulose (or a cellulose derivative), starch (or a starch derivative), and pullulan (or a pullulan derivative), either alone or in combination. Furthermore, plasticizers and hydrocolloid gums may be added to create a gelatin-free capsule composition.

### Method of Producing the Cap and Base

Once the composition of the present invention is prepared according to the general formula set forth above, the composition is melted by heating until a desired viscosity is achieved. The pins of the pin bars are then dipped into the composition and are allowed to cool. The notching described above in relation to the cap petals is undertaken after the caps are cooled but before they are stripped off of the pins. Once notching has been completed, the resulting caps and bases are then stripped from the pins and are assembled with the pledget and are packaged for distribution.

The foregoing discussion discloses and describes an exemplary embodiment of the present invention. One skilled in the art will readily recognize from such discussion, and from the accompanying drawings and claims that various changes, modifications and variations can be made therein without departing from the true spirit and fair scope of the invention as defined by the following claims.

What is claimed is:

1. A method for forming the cap and base for a tampon applicator, the method comprising:
forming a cap pin substantially having the outer shape of the tampon applicator cap;
forming a base pin substantially having the outer shape of the tampon applicator base;
forming a cap and base composition;
heating said composition to a desired viscosity;
dipping each of said cap pin and said base pin into said heated composition;
allowing the coated composition on said cap pin and said base pin to cool; and
removing the cooled cap and base from their respective pins.

2. The method of claim 1 wherein said cap pin has a base and has a plurality of spaced apart peripheral ridges formed adjacent said base.

3. The method of claim 1 wherein said base pin has a base and has a ridge formed adjacent said base.

4. The method of claim 1 wherein said base pin has a plurality of longitudinal grooves formed therein.

5. The method of claim 1 wherein said composition consists of gelatin, silicon dioxide, sodium lauryl sulphate and water to produce a biodegradable product.

6. The method of claim 5 wherein said gelatin is provided in an amount between about 30.00% and 40.00% by weight, said silicon dioxide is provided in an amount of between about 2.00% and 5.00% by weight, said sodium lauryl sulphate is provided in an amount of between about 0.01% and 0.05% by weight, and said water is provided in an amount of between about 67.99% and 54.95% by weight.

7. The method of claim 5 wherein said composition further includes a coloring agent and a preservative.

8. A composition for forming a biodegradable tampon applicator, the composition consisting essentially of: gelatin, silicon dioxide, sodium lauryl sulphate, and water.

9. The composition of claim 8 wherein said gelatin is provided in an amount between about 30.00% and 40.00% by weight, said silicon dioxide is provided in an amount of between about 2.00% and 5.00% by weight, said sodium lauryl sulphate is provided in an amount of between about 0.01% and 0.05% by weight, and said water is provided in an amount of between about 67.99% and 54.95% by weight.

10. The composition of claim 8 further including a coloring agent.

11. The composition of claim 8 further including a preservative.

12. The composition of claim 11 wherein said preservative is methylparaben.

13. A tampon applicator formed from a biodegradable composition, the applicator including a cap and a base, the cap being formed on a cap pin dipped in the biodegradable composition and the base being formed on a base pin dipped in the biodegradable composition.

14. The tampon applicator of claim 13 wherein biodegradable composition consists of gelatin, silicon dioxide, sodium lauryl sulphate and water to produce a biodegradable product.

15. The tampon applicator of claim 14 wherein said gelatin is provided in an amount between about 30.00% and 40.00% by weight, said silicon dioxide is provided in an amount of between about 2.00% and 5.00% by weight, said sodium lauryl sulphate is provided in an amount of between about 0.01% and 0.05% by weight, and said water is provided in an amount of between about 67.99% and 54.95% by weight.

16. The tampon applicator of claim 14 wherein said biodegradable composition further includes a coloring agent.

17. The tampon applicator of claim 14 wherein said biodegradable composition further includes a preservative.

18. The tampon applicator of claim 13 wherein said cap pin has a base and has a plurality of spaced apart peripheral ridges formed adjacent said base.

19. The tampon applicator of claim 13 wherein said base pin has a base and has a ridge formed adjacent said base.

20. The tampon applicator of claim 13 wherein said base pin has a plurality of longitudinal grooves formed therein.
